(19) United States
(12) Patent Application Publication
(10) Pub. No.: US 2023/0383370 A1
(43) Pub. Date: Nov. 30, 2023

(54) METHODS OF SEQUENCING INDIVIDUAL VIRAL GENOMES

(71) Applicant: Bigelow Laboratory for Ocean Sciences, East Boothbay, ME (US)

(72) Inventors: Ramunas Stepanauskas, Nobleboro, ME (US); Greta Leonaviciene, Vilnius (LT)

(73) Assignee: Bigelow Laboratory for Ocean Sciences, East Boothbay, ME (US)

(21) Appl. No.: 18/325,473

(22) Filed: May 30, 2023

Related U.S. Application Data
(60) Provisional application No. 63/347,091, filed on May 31, 2022.

(51) Int. Cl. C12Q 1/70; C12Q 1/6869

(52) U.S. Cl. CPC C12Q 1/701; C12Q 1/6869; C12Q 2600/156; C12Q 1/6806

(57) ABSTRACT

Described herein are methods of sequencing individual viral genomes and methods of determining the viral load of a sample. Also disclosed herein are methods of monitoring the evolution of a viral genome.

### RELATED APPLICATION

This application claims the benefit under 35 U.S.C. 119(e) of U.S. provisional application No. 63/347,091 filed May 31, 2022, the entirety of which is incorporated herein by reference.

### GOVERNMENTAL SUPPORT

This invention was made with Government support under Grant No. 2116253 and Grant No. 1829879, both awarded by the National Science Foundation. The Government has certain rights in the invention.

### BACKGROUND

The COVID-19 pandemic has demonstrated what an enormous toll the emergence of new variants of pathogenic viruses can have on public health, economy and society. Genomic sequencing of viral genomes aids contemporary epidemiology (e.g., by tracking of the emergence of variants) and the development of antiviral vaccines and drugs. Contemporary methods for the genomic sequencing of viruses rely on the bulk extraction and sequencing of viral nucleic acids from a sample.

### SUMMARY

The inventors of the instant application have appreciated that contemporary methods for sequencing viral genomes are not well suited to detect genomic variation among viral particles in a sample and that whole genome sequencing of individual viral genomes in a sample (e.g., an environmental sample or a biological sample, such as a human sample) would be instrumental. Disclosed herein are methods for genomic sequencing of individual extracellular genetic elements (such as individual viral genomes), which are applicable to viral genomes of variable size and samples of high complexity.

Applications of these methods include: genomic sequencing of individual viral particles (including DNA viral genomes and RNA viral genomes) and extracellular, non-viral DNA and RNA molecules from aquatic samples; genomic sequencing of individual viral particles (including DNA viral genomes and RNA viral genomes) and extracellular, non-viral DNA and RNA molecules from a complex sample, such as a sediment sample; genomic sequencing of individual extracellular viral and non-viral particles of single-stranded DNA; genomic sequencing of individual viral particles (including DNA viral genomes and RNA viral genomes) and extracellular, non-viral DNA and RNA molecules from small sample volumes (less than 100 μL); and quantification of extracellular viral and non-viral particles with specific genome sequences.

Using the methods described herein, one is able to recover viral genomes from the environment that are unrecoverable using previously disclosed methods, such as metagenome assembly or FACS-based genomics of individual viral particles. One can also match genomic sequences obtained from individual, free viral particles with viral sequences from infected host cell.

In some aspects, the disclosure relates to a method of sequencing individual viral genomes comprising: (i) encapsulating aliquots of a liquid sample in semi-permeable microcapsules to generate a plurality of encapsulated aliquots collectively comprising a plurality of viral genomes; (ii) exposing the plurality of encapsulated aliquots to amplification conditions to amplify genomic material within the encapsulated aliquots; and (iii) sequencing the amplified genomic material of the encapsulated aliquots. In some embodiments, the semi-permeable microcapsules are semi-permeable hydrogel microcapsules. In some embodiments, the encapsulating of step (i) does not comprise pre-mixing the sample with a dextran phase. In some embodiments, the encapsulating of step (i) comprises supplying the sample and dextran to a microfluidic chip through separate channels.

In some embodiments, the method further comprises separating encapsulated aliquots containing amplified genomic material from encapsulated aliquots lacking amplified genomic material between steps (ii) and (iii). In some embodiments, the aliquots containing amplified genomic material are separated into individual microwells.

In some embodiments, the volume of the aliquots encapsulated by the individual semi-permeable microcapsules is between 1-100 pL.

In some embodiments, the liquid sample is a liquid biological sample. In some embodiments, the liquid biological sample is isolated from a multicellular organism, wherein the multicellular organism is an animal, a fungus, or a plant. In some embodiments, the liquid biological sample comprises blood, saliva, or mucous.

In some embodiments, the liquid sample is a liquid environmental sample. In some embodiments, the liquid environmental sample is a seawater sample, a lake water sample, a river sample, or a wastewater sample.

In some embodiments, the method further comprises enriching for extracellular genetic elements in the liquid sample prior to encapsulating in step (i).

In some embodiments, the method further comprises diluting the liquid sample to increase the probability that no more than one genomic molecule is encapsulated in each semi-permeable microcapsule.

In some embodiments, the liquid sample is cryopreserved and thawed prior to step (i).

In some embodiments, the liquid sample subject to encapsulation in step (i) is a raw liquid sample.

In some embodiments, the liquid sample is generated by dispersing a solid sample into a liquid. In some embodiments, the sample is cryopreserved after dispersal and thawed prior to step (i).

In some embodiments, the solid sample is a solid biological sample. In some embodiments, the solid biological sample is isolated from a multicellular organism, wherein the multicellular organism is an animal, a fungus, or a plant. In some embodiments, the solid biological sample is fecal material or skin.

In some embodiments, the solid sample is a solid environmental sample. In some embodiments, the solid environmental sample is a surface swab, a soil sample, a rock sample, or a marine sediment sample.

In some embodiments, the method further comprises enriching for extracellular genetic elements in the sample prior to encapsulating in step (i). In some embodiments, the enriching comprises separating cellular material from extracellular genetic elements.

In some embodiments, the method further comprises diluting the sample to increase the probability that no more than one genomic molecule is encapsulated in each semi-permeable microcapsule.

In some embodiments, the method comprises flocculation, filtration, flow cytometry, or a combination thereof.

In some embodiments, the method comprises exposing the plurality of encapsulated aliquots to a DNase prior to exposure to amplification conditions in step (ii).

In some embodiments, the method comprises enriching for semi-permeable microcapsules containing amplified genetic elements prior to sequencing in step (iii).

In some embodiments, the method comprises positioning microcapsules containing amplified genetic elements in individual wells of a microplate prior to sequencing in step (iii). In some embodiment, the method further comprises lysing the microcapsules in the individual wells of the microplate. In some embodiments, the method further comprises re-amplifying the amplified genetic elements in the individual wells of the microplate prior to step (iii).

In some embodiments, the method comprises barcoding amplified genetic elements prior to sequencing in step (iii). In some embodiments, the amplified genetic elements are barcoded in individual wells of a microplate.

In some embodiments, the plurality of viral genomes of (i) comprises DNA viral genomes. In some embodiments, the DNA viral genomes comprise single-stranded DNA viral genomes. In some embodiments, the DNA viral genomes comprise genomes that are smaller than 30 kbp.

In some embodiments, the plurality of viral genomes of (i) comprises RNA viral genomes.

In some aspects, the disclosure relates to a method of determining the viral load of a liquid sample comprising: (i) encapsulating aliquots of the liquid sample in individual semi-permeable microcapsules to generate a plurality of encapsulated aliquots, wherein the total volume of liquid sample encapsulated within the random aliquots is known; (ii) exposing the plurality of encapsulated aliquots to amplification conditions to amplify genomic material within the encapsulated aliquots; (iii) sequencing the amplified genomic material of the encapsulated aliquots to identify and quantify individual viral genomes in the plurality of encapsulated aliquots; and (iv) calculating viral load in the sample using the total volume of sample encapsulated within the random aliquots in (i) and the quantity of individual viral genomes of (iii). In some embodiments, the semi-permeable microcapsules are semi-permeable hydrogel microcapsules. In some embodiments, the encapsulating of step (i) does not comprise pre-mixing the sample with a dextran phase. In some embodiments, the encapsulating of step (i) comprises supplying the sample and dextran to a microfluidic chip through separate channels.

In some embodiments, the method further comprises separating encapsulated aliquots containing amplified genomic material from encapsulated aliquots lacking amplified genomic material between steps (ii) and (iii). In some embodiments, the aliquots containing amplified genomic material are separated into individual microwells.

In some embodiments, the volume of the aliquots encapsulated by the individual semi-permeable microcapsules is between 1-100 pL.

In some embodiments, the liquid sample is a liquid biological sample. In some embodiments, the liquid biological sample is isolated from a multicellular organism, wherein the multicellular organism is an animal, a fungus, or a plant. In some embodiments, the liquid biological sample comprises blood, saliva, or mucous.

In some embodiments, the liquid sample is a liquid environmental sample. In some embodiments, the liquid environmental sample is a seawater sample, a lake water sample, a river sample, or a wastewater sample.

In some embodiments, the method further comprises enriching for extracellular genetic elements in the liquid sample prior to encapsulating in step (i).

In some embodiments, the method further comprises diluting the liquid sample to increase the probability that no more than one genomic molecule is encapsulated in each semi-permeable microcapsule.

In some embodiments, the liquid sample is cryopreserved and thawed prior to step (i). In some embodiments, the liquid sample subject to encapsulation in step (i) is a raw liquid sample.

In some embodiments, the liquid sample is generated by dispersing a solid sample into a liquid. In some embodiments, the sample is cryopreserved after dispersal and thawed prior to step (i).

In some embodiments, the solid sample is a solid biological sample. In some embodiments, the solid biological sample is isolated from a multicellular organism, wherein the multicellular organism is an animal, a fungus, or a plant. In some embodiments, the solid biological sample is fecal material or skin.

In some embodiments, the solid sample is a solid environmental sample. In some embodiments, the solid environmental sample is a surface swab, a soil sample, a rock sample, or a marine sediment sample.

In some embodiments, the method further comprises enriching for extracellular genetic elements in the sample prior to encapsulating in step (i). In some embodiments, the enriching comprises separating cellular material from extracellular genetic elements.

In some embodiments, the method further comprises diluting the liquid sample to increase the probability that no more than one genomic molecule is encapsulated in each semi-permeable microcapsule.

In some embodiments, the method comprises flocculation, filtration, flow cytometry, or a combination thereof.

In some embodiments, the method comprises exposing the plurality of encapsulated aliquots to a DNase prior to exposure to amplification conditions in step (ii).

In some embodiments, the method comprises enriching for semi-permeable microcapsules containing amplified genetic elements prior to sequencing in step (iii).

In some embodiments, the method comprises positioning microcapsules containing amplified genetic elements in individual wells of a microplate prior to sequencing in step (iii). In some embodiment, the method further comprises lysing the microcapsules in the individual wells of the microplate. In some embodiments, the method further comprises re-amplifying the amplified genetic elements in the individual wells of the microplate prior to step (iii).

In some embodiments, the method comprises barcoding amplified genetic elements prior to sequencing in step (iii). In some embodiments, the amplified genetic elements are barcoded in individual wells of a microplate.

In some embodiments, the plurality of viral genomes of (i) comprises DNA viral genomes. In some embodiments, the DNA viral genomes comprise single-stranded DNA viral genomes. In some embodiments, the DNA viral genomes comprise genomes that are smaller than 30 kbp.

In some embodiments, the plurality of viral genomes of (i) comprises RNA viral genomes.

In some aspects, the disclosure relates to a method of monitoring the evolution of a viral genome comprising: (i) encapsulating, at a first time point, random aliquots of a first liquid sample in semi-permeable microcapsules to generate a plurality of encapsulated aliquots collectively comprising a plurality of viral genomes; (ii) exposing the plurality of encapsulated aliquots to amplification conditions to amplify genomic material within the encapsulated aliquots; (iii) sequencing the amplified genomic material of the encapsulated aliquots, thereby determining the sequences of the viral genomes in the plurality of viral genomes; (iv) identifying mutations in the viral genomes determined in (iii) by comparing their sequences with those of previously identified viral genomes; and (v) iteratively repeating steps (i)-(iv) at later time points and with additional liquid samples. In some embodiments, the semi-permeable microcapsules are semi-permeable hydrogel microcapsules. In some embodiments, the encapsulating of step (i) does not comprise pre-mixing the sample with a dextran phase. In some embodiments, the encapsulating of step (i) comprises supplying the sample and dextran to a microfluidic chip through separate channels.

In some embodiments, the method further comprises separating encapsulated aliquots containing amplified genomic material from encapsulated aliquots lacking amplified genomic material between steps (ii) and (iii). In some embodiments, the aliquots containing amplified genomic material are separated into individual microwells.

In some embodiments, the volume of the aliquots encapsulated by the individual semi-permeable microcapsules is between 1-100 pL.

In some embodiments, the first liquid sample of (i) and the additional liquid samples of (v) are liquid biological samples. In some embodiments, the liquid biological samples are isolated from multicellular organisms, wherein the multicellular organisms are animals, fungi, or plants. In some embodiments, the liquid biological samples comprise blood, saliva, or mucous.

In some embodiments, the first liquid sample of (i) and the additional liquid samples of (v) are liquid environmental samples. In some embodiments, the liquid environmental samples are seawater samples, lake water samples, river samples, or wastewater samples.

In some embodiments, the method further comprises enriching for extracellular genetic elements in the first liquid sample prior to encapsulating in step (i) and in the additional samples prior to each iterative repetition.

In some embodiments, the method further comprises diluting the first liquid sample and the additional samples prior to encapsulation to increase the probability that no more than one genomic molecule is encapsulated in each semi-permeable microcapsule.

In some embodiments, the first liquid sample and the additional liquid samples are cryopreserved and thawed prior to step (i).

In some embodiments, the first liquid sample and the additional liquid samples that are subject to encapsulation are raw liquid samples.

In some embodiments, the first liquid sample and the additional liquid samples are generated by dispersing a solid sample into a liquid. In some embodiments, the samples are cryopreserved after dispersal and thawed prior to step (i).

In some embodiments, the solid samples are solid biological samples. In some embodiments, the solid biological samples are isolated from multicellular organisms, wherein the multicellular organisms are animals, fungi, or plants. In some embodiments, the solid biological samples are fecal material or skin.

In some embodiments, the solid samples are solid environmental samples. In some embodiments, the solid environmental samples are surface swabs, soil samples, rock samples, or marine sediment samples.

In some embodiments, the method further comprises enriching for extracellular genetic elements prior to encapsulating in step (i). In some embodiments, the enriching comprises separating cellular material from extracellular genetic material.

In some embodiments, the method further comprises diluting the first liquid sample and the additional liquid samples to increase the probability that no more than one genomic molecule is encapsulated in each semi-permeable microcapsule. In some embodiments, the method comprises flocculation, filtration, flow cytometry, or a combination thereof.

In some embodiments, the method comprises exposing the plurality of encapsulated aliquots to a DNase prior to exposure to amplification conditions in step (ii).

In some embodiments, the method further comprises enriching for semi-permeable microcapsules containing amplified genetic elements prior to sequencing in step (iii).

In some embodiments, the method comprises positioning microcapsules containing amplified genetic elements in individual wells of a microplate prior to sequencing in step (iii). In some embodiments, the method further comprises lysing the microcapsules in the individual wells of the microplate. In some embodiments, the method further comprises re-amplifying the amplified genetic elements in the individual wells of the microplate prior to step (iii).

In some embodiments, the method further comprises barcoding amplified genetic elements prior to sequencing in step (iii). In some embodiments, the amplified genetic elements are barcoded in individual wells of a microplate.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 provides a schematic representation of a preferred embodiment of the methods described herein. The depicted method comprises the following steps: 1) generation of semi-permeable hydrogel microcapsules filled with random micro-aliquots of the analyzed sample; 2) cDNA generation and/or DNA amplification inside capsules; 3) separation of individual microcapsules that contain amplified DNA into microplate wells; and 4) sequencing and de novo assembly of DNA from individual microcapsules. The representation of the seawater microbial environment is borrowed from (9).

FIG. 2 provides a schematic representation of an exemplary method for genomic sequencing of DNA viral particles and other DNA-containing particles. The depicted method comprises the following steps: i) optionally cryopreserving (and subsequently thawing) a sample; ii) optionally enriching for extracellular genetic elements (e.g., by removing cellular material) by filtration or centrifugation; iii) generating semi-permeable hydrogel microcapsules filled with random micro-aliquots of the sample; iv) optionally exposing the encapsulated micro-aliquots to a DNase (to digest naked DNA inside the microcapsules); v) lysing viral particles and amplifying genomic DNA inside the microcapsules; vi) isolating individual microcapsules that contain amplified DNA into microplate wells; vii) lysing microcapsules, optionally re-amplifying genomic DNA, and creating barcoded sequencing libraries inside the microplate wells; and viii) sequencing and de novo assembling of gDNA from individual microcapsules.

FIG. 3 provides a schematic representation of an exemplary method for genomic sequencing of RNA viral particles and other RNA-containing particles. The depicted method comprises the following steps: i) optionally cryopreserving (and subsequently thawing) a sample; ii) optionally enriching for extracellular genetic elements (e.g., by removing cellular material) by filtration or centrifugation; iii) generating semi-permeable hydrogel microcapsules filled with random micro-aliquots of the sample; iv) lysing viral particles and exposing the encapsulated micro-aliquots to a DNase; v) amplifying genomic RNA by reverse transcription and cDNA amplification inside the microcapsules; vi) isolating individual microcapsules that contain amplified cDNA into microplate wells; vii) lysing microcapsules, optionally re-amplifying cDNA, and creating barcoded cDNA sequencing libraries inside microplate wells; and viii) sequencing and de novo assembling of cDNA from individual microcapsules.

FIGS. 4A-4D relate to the recovery of viral DNA sequences from seawater samples from the Gulf of Maine. Samples were prepared as described in Example 2. FIG. 4A shows the fraction of genes in de novo genome assemblies assigned to “host” (non-viral) sources. FIG. 4B shows de novo genome assembly length. FIG. 4C shows the count of viral contigs with diverse quality scores. Bars from left to right are: complete; high quality; medium quality; low quality; and undetermined. FIG. 4D shows recruitment of reads from the Gulf of Maine DNA viral metagenome on diverse reference libraries.

FIGS. 5A-5C relate to the recovery of viral DNA sequences from a coastal sediment sample from the Gulf of Maine. Samples were prepared as described in Example 3. FIG. 5A shows the fraction of genes in de novo genome assemblies assigned to “viral”, “microbial” (non-viral) and “unknown” sources. Bars from left to right are: viral; microbial; and unknown. FIG. 5B. shows the count of genome assemblies identified as “viral” with diverse quality scores. Bars from left to right are: high quality; medium quality; low quality; and not determined. FIG. 5C. shows the de novo genome assembly length.

FIGS. 6A-6B relate to the recovery of viral DNA sequences from deep ocean seawater samples. Samples were prepared as described in Example 4. Genome sequences of individual viral particles that have homologous regions (average amino acid identity ≥90%) to infecting agents recovered from single cells of host bacterial and archaeal cells were identified. FIG. 6A shows exemplary viral particle microcapsule SAGs having regions of similarity to a Pelagibacterales (Alphaproteobacteria) FACS SAG. FIG. 6B shows exemplary viral particle microcapsule SAGs having regions of similarity to a Nitrososphaerales (Thermoproteota) FACS SAG.

FIG. 7 provides an example of the genome content of a microcapsule SAG AM-555-F03 that has been determined as a viral genome. This SAG was prepared according to the method d) of Example 2. Using BLASTn, no matching DNA sequences in the metagenome assembly prepared from the same sample were identified. This demonstrates the capacity of microcapsule SAGs to discover genomic sequences that cannot be discovered using prior methods.

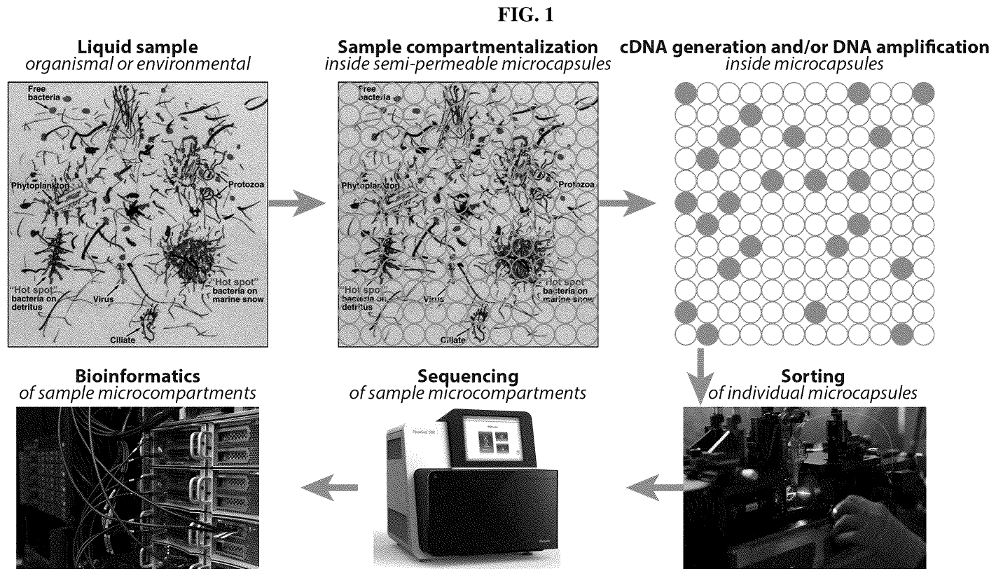

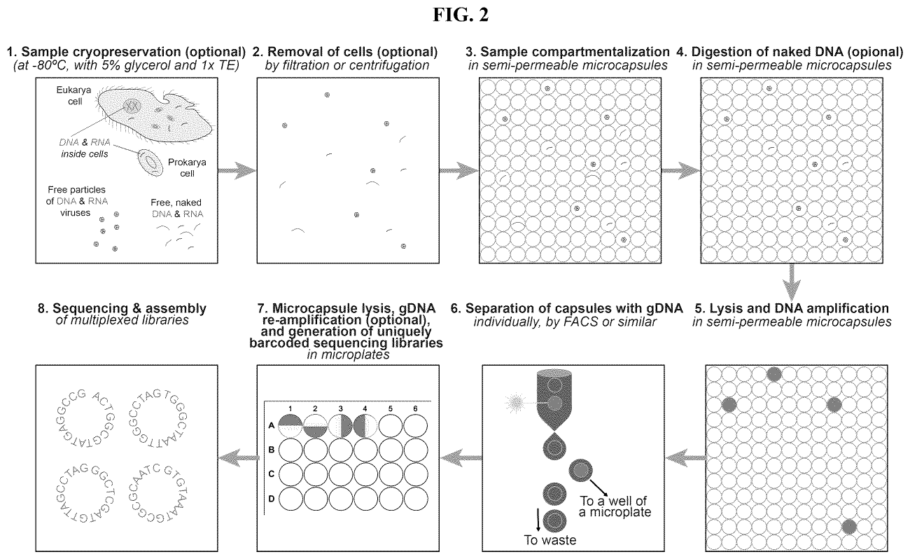

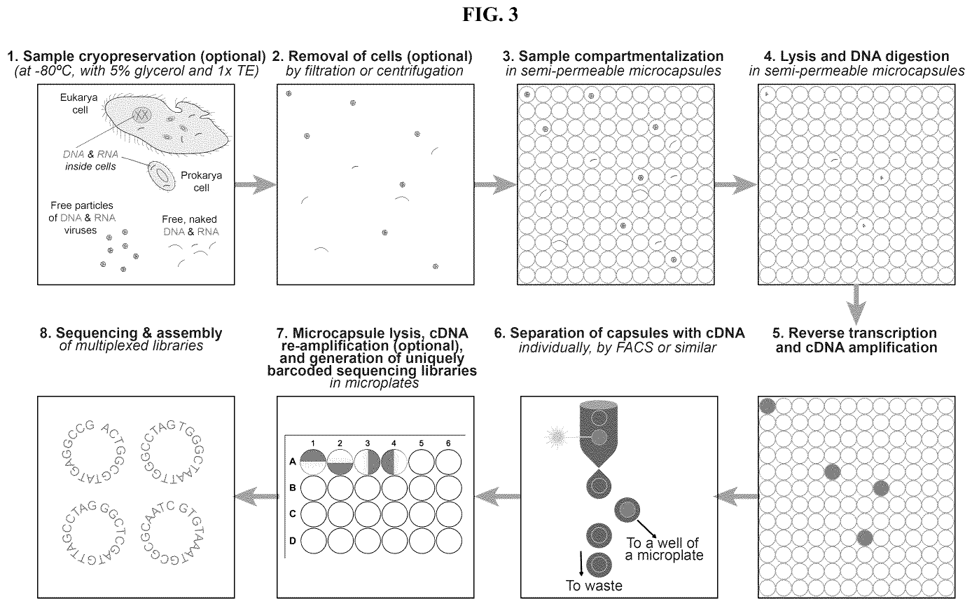

### DETAILED DESCRIPTION

Contemporary methods for the genomic sequencing of viruses rely on the bulk extraction and sequencing of viral nucleic acids from a sample. This approach is not well suited to detect genomic variation among viral particles in a sample. Moreover, results from environmental viral metagenomics show that these bulk analyses are prone to biases introduced during DNA and RNA collection, extraction, sequencing, and computation analyses (1-4).

Assembly-free recovery of entire viral genomes has been reported using nanopore sequencing technology, which relies on single molecule sequencing and eliminates the need for de-novo assembly and the associated assembly fragmentation, chimerism and loss of repeat regions. However, this nanopore sequencing technology is limited to double-stranded DNA within a size range of approximately 30-90 kilobases. As viruses can have double- or single-stranded DNA or RNA, and their known size range spans from a few kilobases to several megabases, the diversity of viral genomes that can be recovered using the nanopore technique is very limited.

Like the nanopore sequencing technique, flow cytometry-mediated single virus genomics, which has been demonstrated on seawater samples (6-8), also enables the sequencing of individual viral genomes. Flow cytometry sorting techniques have their drawbacks, however, due to the necessary step of gating DNA-containing particles. Viruses are at the very lower limit of detection of flow cytometers, thus there are risks that one might miss small viruses, gate-out larger viruses, and overwhelm the analytical pipeline with false positives, such as non-biological particles or electronic instrument noise.

Although publications pertaining to genomic sequencing of individual DNA viruses exist, the reported methods are not suitable for small DNA viruses (e.g., papillomavirus), RNA viruses (e.g., COVID-19) or complex samples (such as sediments, soils, human blood, saliva, swabs or fecal material).

Disclosed herein are methods that address these shortcomings. These methods utilize: (i) encapsulation of individual genetic elements in semi-permeable microcapsules (e.g., semi-permeable hydrogel microcapsules); (ii) amplification of genetic elements encapsulated within the semi-permeable microcapsules; and (iii) sequencing of amplified genetic elements. In some embodiments, the methods further comprise: cryopreserving the sample prior to step (i); enriching for extracellular genetic elements in a sample and/or diluting a liquid sample prior to encapsulating in step (i); DNase treatment after encapsulating in step (i); enriching for semi-permeable microcapsules containing amplified genetic elements prior to sequencing in step (iii); DNA re-amplification prior to sequencing in step (iii); and/or barcoding amplified genetic elements prior to sequencing in step (iii).

### I. Exemplary Samples

The methods described herein are applicable to a wide range of sample sources, including complex sample sources.

In some embodiments, a sample is a biological sample. A biological sample may be isolated from a multicellular organism, such as an animal, a fungus, or a plant. In some embodiments, a biological sample is isolated from a human (e.g., a human patient). In some embodiments, a biological sample comprises blood, saliva, mucous, fecal material, or skin.

In some embodiments, a sample is an environmental sample. In some embodiments, an environmental sample comprises seawater, lake water, river water, wastewater, sediment, or soil.

To be encapsulated within semi-permeable microcapsules (e.g., semi-permeable hydrogel microcapsules), a sample is preferably in a liquid form. As such, in some embodiment, a sample is a liquid sample (e.g., a liquid biological sample or a liquid environmental sample). In some embodiments, a solid sample (e.g., a solid biological sample or a solid environmental sample) is converted into a liquid sample prior to encapsulation within semi-permeable microcapsules (e.g., semi-permeable hydrogel microcapsules). In some embodiments, a solid sample is converted to a liquid sample by dispersing the solid sample into a liquid; for example, at a ratio (solid:liquid) of 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20. Exemplary liquids that may be used to disperse a solid sample include, but are not limited to laboratory-made aquatic buffers (e.g. phosphate-buffered saline, tris-EDTA buffer) and natural aquatic solutions (e.g. lake or sea water) that have been autoclaved and/or decontaminated (e.g. by exposure to UV) from polymerase-amplifiable nucleic acids. In some embodiments, a dispersed solid sample is vortexed, e.g., for at least 1 second, at least 5 seconds, at least 10 seconds, at least 20 seconds, at least 25 seconds, at least 30 seconds, or at least 1 minute.

In some embodiments, a sample (a liquid sample or a dispersed solid sample) has a volume of less than 1000 μL, less than 900 μL, less than 800 μL, less than 700 μL, less than 600 μL, less than 500 μL, less than 400 μL, less than 300 μL, less than 200 μL, less than 190 μL, less than 180 μL, less than 170 μL, less than 160 μL, less than 150 μL, less than 140 μL, less than 130 μL, less than 120 μL, or less than 110 μL. In some embodiments a sample (liquid sample or dispersed solid sample) has a volume of 100-1000 μL, 100-900 μL, 100-800 μL, 100-700 μL, 100-600 μL, 100-500 μL, 100-400 μL, 100-300 μL, 100-200 μL, 100-190 μL, 100-180 μL, 100-170 μL, 100-160 μL, 100-150 μL, 100-140 μL, 100-130 μL, 100-120 μL, or 100-110 μL.

### II. Sample Storage/Cryopreservation

In some embodiments, a sample is prepared for genomic sequencing fresh (i.e., without storage). In some embodiments, a sample is cryopreserved after the sample is isolated and before the sample is prepared for genomic sequencing. In some embodiments, cryopreservation consists of 5% glycerol and 1×pH 8 TRIS-EDTA buffer amendments (final concentrations) followed by storage at −70° C. or lower temperature for a period of time spanning from one hour to multiple years. Indeed, the inventors have unexpectedly found that sample cryopreservation prior to processing enhances recovery of viral DNA.

III. Enriching and/or Diluting a Sample Prior to Encapsulation

In some embodiments, a sample is isolated from its source (e.g., its biological source or its environmental source) and directly subject to encapsulation within semi-permeable microcapsules, such as semi-permeable hydrogel microcapsules (e.g., without enrichment or dilution). A sample isolated from its source and directedly subjected to encapsulation is referred to herein as a “raw” sample (e.g., a raw liquid sample).

In other embodiments, however, a method comprises enriching a sample for extracellular genetic elements and/or diluting a sample to increase the probability that no more than one genomic molecule is encapsulated within a semi-permeable microcapsule (e.g., semi-permeable hydrogel microcapsule).

As used herein, the term “genetic element” refers to a polynucleotide that encodes for at least a portion of a genome.

As used herein, the term “extracellular genetic element” refers to a polynucleotide located outside of a cell that encodes for at least a portion of a genome. Exemplary extracellular genetic elements include viral genomes (or portions thereof) that are not within a cell, bacteriophage genomes (or portions thereof) that are not within a cell, plasmids (or portions thereof) that are not within a cell (e.g., from a ruptured cell), and chromosomes (or portions thereof) that are not with a cell (e.g., from a ruptured cell). As such, the individual extracellular genetic elements that are encompassed by the methods described herein comprise viral genomes (or portions thereof), bacteriophage genomes (or portions thereof), plasmids (or portions thereof), extracellular vesicles (or portions thereof), and chromosomes (or portions thereof).

In some embodiments described herein, an extracellular genetic element is a viral genome (or a portion thereof). These viral genomes include RNA genomes and DNA viral genomes of variable size (i.e., from a few kilobases to several megabases). As a result, the methods described herein are applicable to analyses of DNA viruses smaller than approximately 30 kilobases (e.g., papillomavirus), DNA viruses larger than approximately 90 kilobases (e.g., herpes simplex), and RNA viruses (e.g., COVID-19).

A. Enriching a Sample for Extracellular Genetic Elements In some embodiments, a method described herein comprises a step of enriching a sample for extracellular genetic elements. A process of enriching may comprise filtration, centrifugation, immunomagnetic separation, or a combination thereof.

In some embodiments, enriching for extracellular genetic elements comprises passing a liquid sample (or a solid sample that is converted into a liquid sample) through a filter with a pore size that is too small for most cells to pass, thereby reducing the insoluble material found within the liquid sample. In some embodiments, the filter has an average pore size of between 0.1-30 μm, 0.1-25 μm, 0.1-20 μm, 0.1-15 μm, 0.1-10 μm, 0.1-9 μm, 0.1-8 μm, 0.1-7 μm, 0.1-6 μm, 0.1-5 μm, 0.1-4 μm, 0.1-3 μm, 0.1-1 μm, 0.1-0.9 μm, 0.1-0.8 μm, 0.1-0.7 μm, μm, 0.1-0.5 μm, 0.1-0.4 μm, 0.1-0.3 μm, or 0.1-0.2 μm. In some embodiments, the filter has an average pore size of about 0.1 μm. In some embodiments, the filter has an average pore size of about 0.2 μm.

In some embodiments, enriching for extracellular genetic elements comprises subjecting a liquid sample (or a solid sample that is converted into a liquid sample) to centrifugation and then retaining the supernatant, thereby reducing the insoluble material found within the liquid sample.

In some embodiments, enriching for extracellular genetic elements comprises exposing a liquid sample (or a solid sample that is converted into a liquid sample) to affinity beads (e.g., immunomagnetic beads). In some embodiments, the affinity beads bind to one or more biomolecules found on the surface of a cell, thereby reducing the cellular content of the sample. In some embodiments, the affinity beads bind to polynucleic acids, and the method further comprises: isolating the beads; eluting the polynucleic acids from the beads to generate an elution sample; and encapsulating the eluted sample within semi-permeable microcapsules (e.g., semi-permeable hydrogel microcapsules).

B. Diluting a Sample

In some embodiments, a method comprises diluting a sample to increase the probability that no more than one genomic molecule is encapsulated within a semi-permeable microcapsule (e.g., a semi-permeable hydrogel microcapsule). In some embodiments, a liquid sample (or a solid sample converted to a liquid sample) is diluted prior to enrichment. In some embodiments, a liquid sample (or a solid sample converted to a liquid sample) is diluted after enrichment. In some embodiments, a liquid sample (or a solid sample converted to a liquid sample) is diluted in the absence of enrichment.

Fluids that can be used to dilute a liquid sample (or a solid sample converted to a liquid sample) include, but are not limited to, laboratory-made aquatic buffers (e.g., phosphate-buffered saline, tris-EDTA buffer) and natural aquatic solutions (e.g., lake or sea water) that have been decontaminated (e.g., by exposure to UV) from polymerase-amplifiable nucleic acids.

### IV. Encapsulation of Individual Genetic Elements in Semi-Permeable Microcapsules

The methods described herein include a step of encapsulating individual extracellular genetic elements of a sample in semi-permeable microcapsules (e.g., semi-permeable hydrogel microcapsules). Methods of generating semi-permeable microcapsules and of encapsulating aliquots of a sample within semi-permeable microcapsules have been described previously (13). Yet, in contrast to the previously described applications of semi-permeable microcapsules (which enriched for cellular genetic elements—i.e., genetic elements found with in a cell—by isolating and washing cells prior to encapsulation), the methods disclosed herein enable the genomic analysis of (and in some embodiments the quantification of) individual extracellular genetic elements found within a sample. Indeed, to increase the probability that a semi-permeable microcapsule comprises an extracellular genetic element found within the original sample, steps are not taken to enrich for cellular genetic elements (i.e., genetic elements found within a cell at the time of sample isolation). For example, in some embodiments extracellular genetic elements are enriched for as described above. Moreover, as cell lysis after sample isolation could introduce genetic elements into the sample after its isolation from its source, in some embodiments, a sample is not exposed to conditions that would lyse a cell prior to enriching for extracellular genetic elements (as described above) or, in some embodiments, prior to encapsulation.

As used herein, the term “semi-permeable microcapsule” refers to a capsule containing a thin, semi-permeable shell which acts as a passive sieve, which retains large molecular weight compounds (such as genomic DNA and RNA) while allowing smaller molecules (such as proteins) to diffuse through. In some embodiments, the semi-permeable microcapsules utilized herein (e.g., semi-permeable hydrogel microcapsules) have an average pore size of 10-100 nm, 10-90 nm, 10-80 nm, 10-70 nm, 10-60 nm, 10-50 nm, 10-40 nm, 10-nm, 20-100 nm, 20-90 nm, 20-80 nm, 20-70 nm, 20-60 nm, 20-50 nm, 20-40 nm, or 20-30 nm. In some embodiments, the semi-permeable microcapsules utilized herein (e.g., semi-permeable hydrogel microcapsules) have volumes of between 1-200 pL, 1-10 pL, 1-9 pL, 1-8 pL, 1-7 pL, 1-6 pL, 2-10 pL, 2-9 pL, 2-8 pL, 2-7 pL, 2-6 pL, 3-10 pL, 3-9 pL, 3-8 pL, 3-7 pL, 3-6 pL, 4-10 pL, 4-9 pL, 4-8 pL, 4-7 pL, 4-6 pL, 5-10 pL, 5-9 pL, 5-8 pL, 5-7 pL, 5-6 pL, 10-100 pL, 20-100 pL, 30-100 pL, 40-100 pL, 50-100 pL, 60-100 pL, 70-100 pL, 80-100 pL, 90-100 pL, 10-90 pL, 10-80 pL, 10-70 pL, 10-60 pL, 10-50 pL, 10-40 pL, 10-30 pL, or 10-20 pL. In some embodiments, the semi-permeable microcapsules (e.g., semi-permeable hydrogel microcapsules) have average volumes of about 1 pL, 2 pL, 3 pL, 4 pL, 5 pL, 6 pL, 7 pL, 8 pL, 9 pL, 10 pL, about 15 pL, about 15 pL, about 20 pL, about 25 pL, about 30 pL, about 35 pL, about 40 pL, about 45 pL, about 50 pL, about 55 pL, about 60 pL, about 65 pL, about 70 pL, about 75 pL, about 80 pL, about 85 pL, about 90 pL, about 95 pL, or about 100 pL. In some embodiments, the semi-permeable microcapsules (e.g., semi-permeable hydrogel microcapsules) have average volumes of about 5 to about 6 pL. In some embodiments, the semi-permeable microcapsules (e.g., semi-permeable hydrogel microcapsules) have average volumes of about 5.5 pL.

In some embodiments, a semi-permeable microcapsule is a semi-permeable hydrogel microcapsule. As used herein, the term “semi-permeable hydrogel microcapsule” refers to hydrogel particles comprising dextran-rich cores and polyethylene glycol diacrylate (PEGDA)-rich shells, which retain large molecule weight compounds, such as DNA/RNA (e.g., comprising a viral genome, or a portion thereof), while permitting the passage of smaller compounds, such as analytical reagents.

In some embodiments, the semi-permeable hydrogel microcapsules used in the methods described herein have cores comprising 5%-15%, 6%-15%, 7%-15%, 8%-15%, 9%-15%, 5%-14%, 5%-13%, 5%-12%, 5%-11%, 6%-13%, 7%-13%, 8%-12%, 9%-11%, or 9-10% (w/v) dextran (MW 500K). In some embodiments, the cores comprise about 8%, about 9%, about 10%, about 11%, or about 12% (w/v) dextran (MW 500K). In some embodiments, the cores have diameters of 25-35 μm, 26-34 μm, 27-33 μm, 28-32 μm, or 29-31 μm.

In some embodiments, the semi-permeable hydrogel microcapsules used in the methods described herein have shells comprising 6%-16%, 7%-16%, 8%-16%, 9%-16%, 10%-16%, 6%-15%, 6%-14%, 6%-13%, 6%-12%, 7%-15%, 8%-14%, 9%-13%, or 10%-12% (w/v) PEGDA. In some embodiments, the shells comprise about 9%, about 10%, about 11%, about 12%, or about 13% (w/v) PEGDA. In some embodiments, shells comprise blends of longer and shorter PEGDA polymers. For example, in some embodiments, the shells comprise blends of MW 8K and MW 575K PEGDA polymers. In some embodiments, the shells have thicknesses of 1-6 μm, 1.5-5.5 μm, 2-5 μm, or 2.5-4.5 μm.

In some embodiments, the semi-permeable hydrogel microcapsules used in the methods described herein further comprise lithium phenyl-2,4,6-trimethylbenzoylphosphinate (LAP).

In some embodiments, the semi-permeable hydrogel microcapsules used in the methods described herein further comprise a buffering agent, such as phosphate buffered saline.

In some embodiments, a semi-permeable microcapsule is a semi-permeable gelatin microcapsule.

In some embodiments, a semi-permeable microcapsule is a semi-permeable agarose microcapsule.

### V. DNase Treatment

In some embodiments, encapsulated samples are exposed to a DNase. In some embodiments, the DNase is dsDNase (ThermoFisher Scientific, catalogue number EN0771).

### VI. Amplification of Genetic Elements Encapsulated Within Semi-Permeable Microcapsules.

The methods described herein include a step of amplifying genetic elements encapsulated with semi-permeable microcapsules (e.g., semi-permeable hydrogel microcapsules). Methods of amplifying genetic elements within semi-permeable microcapsules have been described previously (13).

The conditions for amplifying genetic elements (or “amplification conditions”) within semi-permeable microcapsules (e.g., semi-permeable hydrogel microcapsules) comprise a polymerase, primers, nucleotides, and a reaction buffer.

In some embodiments, the amplification conditions comprise an RNA polymerase. In some embodiments, the amplification conditions comprise a DNA polymerase. In some embodiments, the amplification conditions comprise an RNA polymerase and a DNA polymerase. In some embodiments, the semi-permeable microcapsules (e.g., semi-permeable hydrogel microcapsules) are exposed to the RNA polymerase prior to exposure to the DNA polymerase.

In some embodiments, the amplification conditions comprise random primers. In some embodiments, a primer comprises a nucleic acid sequence of a sequencing primer and/or a barcode, which can be used for downstream sequencing purposes.

In some embodiments, the amplification is performed using WGA-X (7).

In some embodiments, the amplification contains positive and negative controls for quality control.

### VII. Enriching for Semi-Permeable Microcapsules Containing Amplified Genetic Elements

In some embodiments, the methods described herein include a step of enriching for semi-permeable microcapsules (e.g., semi-permeable hydrogel microcapsules) containing amplified genetic elements prior to the sequencing of genetic elements encapsulated with semi-permeable microcapsules. Methods of enriching include but are not limited to: fluorescence-activated cell sorting, on-chip microfluidic sorting, and manual selection.

In some embodiments, semi-permeable microcapsules (e.g., semi-permeable hydrogel microcapsules) containing amplified genetic material are separated from semi-permeable microcapsules lacking amplified genetic material using FACS sorting. In some embodiments, the semi-permeable microcapsules are contacted with a stain that intercalates with DNA (e.g., SYBR Green I dye, SYTO-9 dye) prior to FACS sorting.

In some embodiments, semi-permeable microcapsules (e.g., semi-permeable hydrogel microcapsules) containing amplified genomic material are separated into individual microwells of a plate. In some embodiments, the method further comprises lysing the microcapsules in the individual wells of the microplate. In some embodiments, lysis is performed by exposure to KOH. In some embodiments, lysis is performed as previously described for microbial cells (7). In some embodiments, the method further comprises re-amplifying the amplified genetic elements in the individual wells of the microplate prior to step (iii). In come embodiments, DNA re-amplification is performed by WGA-X (7).

### VIII. Barcoding Amplified Genetic Elements

In some embodiments, barcoded sequencing libraries of amplified genetic elements are created (e.g., after enriching for semi-permeable microcapsules containing amplified genetic elements). In some embodiments, barcoded libraries are created with Nextera XT (Illumina) reagents. In some embodiments, barcoded libraries are created as previously described (7).

### IX. Sequencing of Amplified Genetic Elements

The methods described herein include a step of sequencing amplified genetic elements. Methods of sequencing individual extracellular genetic elements have been described previously (7).

In some embodiments, the amplified genetic elements are sequenced in parallel.

In some embodiments, sequencing libraries of each genetic element have unique DNA barcodes.

In some embodiments, sequencing libraries with unique DNA barcodes are sequenced in a single, pooled batch (multiplexed).

In some embodiments, reads of multiplexed sequencing libraries are demultiplexed, de novo assembled and annotated using state of the art tools, such as bcl2fastq (Illumina Corporation), SPAdes (14) and prokka (15).

### X. Exemplary Methods Encompassed by the Methods Described Herein

In some aspects, the disclosure relates to methods of sequencing individual viral genomes. In some embodiments, the method comprises: (i) encapsulating aliquots of a liquid sample in semi-permeable microcapsules (e.g., semi-permeable hydrogel microcapsules) to generate a plurality of encapsulated aliquots collectively comprising a plurality of viral genomes; (ii) exposing the plurality of encapsulated aliquots to amplification conditions to amplify genomic material within the encapsulated aliquots; and (iii) sequencing the amplified genomic material of the encapsulated aliquots.

In other aspects, the disclosure relates to methods of determining the viral load of a sample. In some embodiments, a method of determining the viral load of a liquid sample (or a solid sample converted to a liquid sample) comprises: (i) encapsulating aliquots of the liquid sample in individual semi-permeable hydrogel microcapsules (e.g., semi-permeable hydrogel microcapsules) to generate a plurality of encapsulated aliquots, wherein the total volume of liquid sample encapsulated within the random aliquots is known; (ii) exposing the plurality of encapsulated aliquots to amplification conditions to amplify genomic material within the encapsulated aliquots; (iii) sequencing the amplified genomic material of the encapsulated aliquots to identify and quantify individual viral genomes in the plurality of encapsulated aliquots; and (iv) calculating viral load in the sample using the total volume of sample encapsulated within the random aliquots in (i) and the quantity of individual viral genomes of (iii).

In some embodiments, the step of identifying individual viral genomes comprises comparing the sequences of the amplified genomic material with the sequences of known viral genomes. It is understood that the sequences of at least a subset of the amplified genomic material in (iii) will include only a partial sequence of a viral genome (e.g., because the genetic element was partially degraded at the time of its isolation). In some embodiments, the step of quantifying individual viral genomes only considers amplified genomic material sequences that comprise a complete viral genome sequence. In other embodiments, the step of quantifying individual viral genomes considers amplified genomic material sequences that comprise a complete viral genomes, as well as amplified genomic material sequences that include only partial viral genomes (e.g., amplified genomic material sequences having—across their length—at least about 90%, at least about 95%, or at least about 99% identity to a known viral genome and containing at least 25%, at least 30%, at least 35%, at least 40%, at least 45%, at least 50%, at least 60%, at least 70%, at least 80%, at least 85%, at least 85%, at least 90%, at least 95%, or at least 99% of the complete genome sequence).

In other aspects, the disclosure relates to methods of monitoring the evolution of a viral genome. In some embodiments, a method of monitoring the evolution of a viral genome comprises: (i) encapsulating, at a first time point, random aliquots of a first liquid sample (or a first solid sample that has been converted to a liquid sample) in semi-permeable microcapsules (e.g., semi-permeable hydrogel microcapsules) to generate a plurality of encapsulated aliquots collectively comprising a plurality of viral genomes; (ii) exposing the plurality of encapsulated aliquots to amplification conditions to amplify genomic material within the encapsulated aliquots; (iii) sequencing the amplified genomic material of the encapsulated aliquots, thereby determining the sequences of the viral genomes in the plurality of viral genomes; (iv) identifying mutations in the viral genomes determined in (iii) by comparing their sequences with those of previously identified viral genomes; and (v) iteratively repeating steps (i)-(iv) at later time points and with additional liquid samples (or additional solid samples that have been converted to liquid samples).

It is understood that the sequences of at least a subset of the amplified genomic material in (ii) will include only a partial sequence of a viral genome (e.g., because the genetic element was partially degraded at the time of its isolation). In some embodiments, the step of identifying mutations only considers amplified genomic material sequences that comprise a complete viral genome sequence. In other embodiments, the step of identifying mutations considers amplified genomic material sequences that comprise a complete viral genomes, as well as amplified genomic material sequences that include only partial viral genomes (e.g., amplified genomic material sequences having—across their length—at least about 90%, at least about 95%, or at least about 99% identity to a known viral genome and containing at least 25%, at least 30%, at least 35%, at least 40%, at least 45%, at least 50%, at least 60%, at least 70%, at least 80%, at least 85%, at least 85%, at least 90%, at least 95%, or at least 99% of the complete genome sequence).

In some embodiments, steps (i)-(iv) are iteratively repeated at least 3 times, at least 4 times, at least 5 times, at least 6 times, at least 7 times, at least 8, times at least 9 times, or at least 10 times.

In some embodiments, the period of time between one or more iterative repetitions is at least 1 week, at least 2 weeks, at least 3 weeks, at least 1 month, at least 2 months, at least 3 months, at last 4 months, at least 5 months, at least 6 months, or at least 1 year.

### EXAMPLES

### Example 1: Sequencing of Individual DNA Viral Genomes from Seawater and Marine Sediment Samples

As a proof of principle, individual DNA viral genomes from seawater samples and marine sediments samples were sequenced. Techniques were optimized for pre-analytical handling of samples, proving that liquid samples can be successfully cryopreserved at −80° C. after an amendment of 5% glycerol and 1×pH 8 TRIS-EDTA buffer (final concentrations). A comparison of particle lysis techniques was performed, which included lysis using KOH (7), lysozyme-triton-x-EDTA (13), proteinaseK-SDS (13), and guanidinium thiocyanate. We determined the following, previously described (7) lysis procedure to be the simplest and the most effective: amendment with 400 mM KOH, 10 mM EDTA and 100 mM dithiothreitol (final concentrations) and a subsequent 10-min incubation at 20° C. The lysis is terminated by the addition of 350 mM Tris-HCl, pH 4 (final concentration).

An ONYX (Droplet Genomics) microfluidic system was used to capture tiny (few picoliters), random parcels of raw sample inside microcapsules composed of polymers that are permeable to DNA amplification reagents but not to DNA (13). The small volume of each microcapsule (range ˜1-100 pL) and appropriate sample dilution ensured minimal probability that more than one DNA-containing particle is co-incapsulated. The randomized micro-encapsulation and sequencing of sample microaliquots means that optical particle detection or capture by flocculation or filtration are no longer needed, thus eliminating associated methodological biases. Subsequent bioinformatic analyses of each genome assembly provide individual particle identities.

### Example 2: Recovery of Viral DNA Sequences from Seawater Sample

Gulf of Maine seawater samples were collected from 1 m depth on Apr. 18, 2022, in Boothbay Harbor (43.84° N, 69.64° W). Four methods of preparing seawater samples for genomic sequencing were compared:
a) Fluorescence-activated cell sorting (FACS)-based single cell genomics targeting Prokarya cells.
b) FACS-based single particle genomics targeting individual viral particles.
c) Microcapsules generated by: (i) generation of semi-permeable hydrogel microcapsules filled with random micro-aliquots of the sample; (ii) viral particle lysis and genomic DNA amplification inside the microcapsules; (iii) separation of individual microcapsules that contain amplified DNA into microplate wells; (iv) lysis of microcapsules and a second round of DNA amplification in microplate wells; and (v) production of uniquely barcoded sequencing libraries inside microplate wells. The sample was processed fresh, on the day of collection.
d) Microcapsules generated by: (i) cryopreservation of the sample; (ii) generation of semi-permeable hydrogel microcapsules filled with random micro-aliquots of the sample (after thawing the sample); (iii) viral particle lysis and genomic DNA amplification inside the microcapsules; (iv) separation of individual microcapsules that contain amplified DNA into microplate wells; (v) lysis of microcapsules and a second round of DNA amplification in microplate wells; and (vi) production of uniquely barcoded sequencing libraries inside microplate wells.

The generation of FACS-based single amplified genomes (SAGs) of prokaryote cells and individual viral particles in methods a) and b), respectively, was performed as previously described (7).

The encapsulation of field samples in methods c) and d) was performed with the Onyx instrument (Droplet Genomics, Vilnius, Lithuania) using PEGDA chemistry, as described previously (13), except that the sample and dextran phases were not pre-mixed and, instead, were supplied to the microfluidic chip through separate channels. The latter method modification was made to eliminate the risk of dextran inducing cell lysis or other interference with the sample prior to encapsulation. Prior to encapsulation, all samples were passed through a 40 μm mesh-size filter. The following flow rates were applied on the microcapsule generation chip: sample, 35 μL h-1; PEGDA, 40 μL h-1; dextran, 30 μL h-1; oil, 500 μL h-1.

Particle and microcapsule lysis and DNA amplification inside microcapsules and inside microplate wells in methods a)-d) were performed following the same protocol as for the FACS-processed individual cells and viral particles (7).

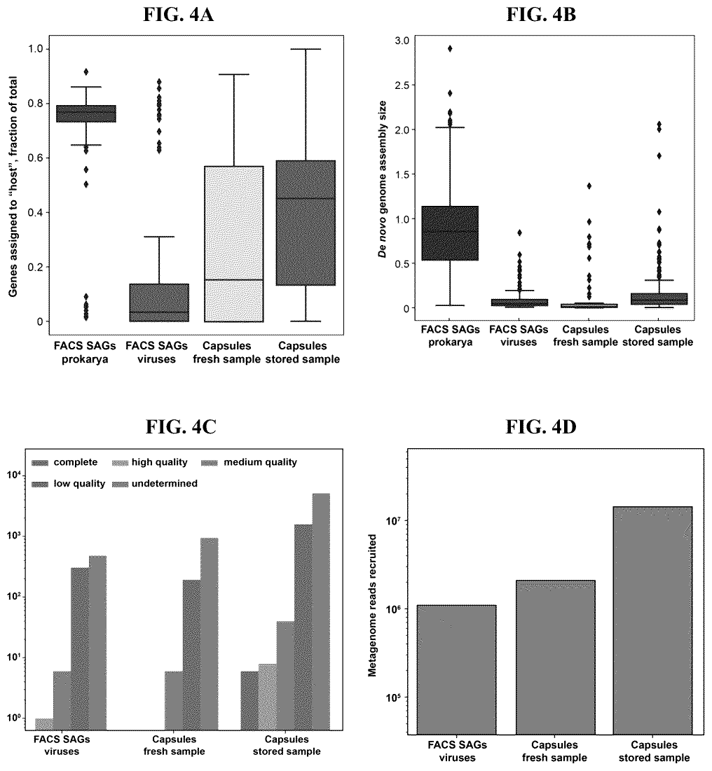

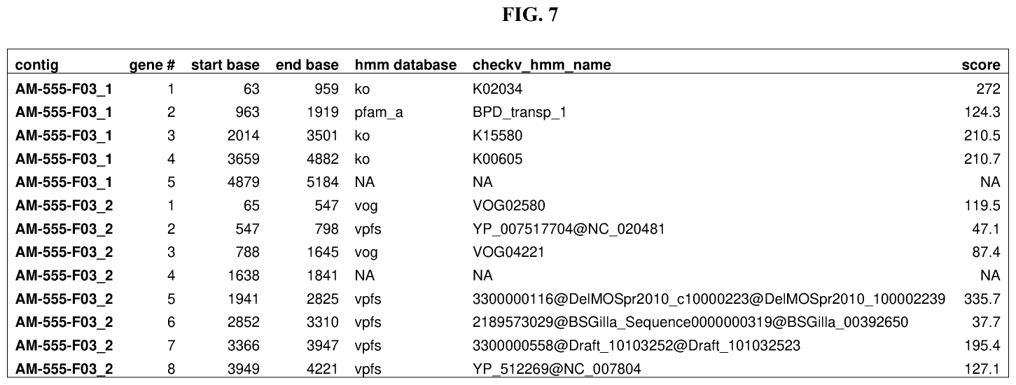

The sequencing libraries for methods a)-d) were then sequenced, de novo assembled and annotated as previously described (7). Detection of virus- and host-associated genes in an assembly was performed with CheckV (17) and VirSorter2 (16). Results are shown in FIGS. 4A-4D and FIG. 7.

Microcapsule-based single amplified genome (SAG) libraries generated according to methods c) and d) were depleted in genes assigned to “host” genomes and, in turn, enriched in viral genes, relative to FACS-based SAG libraries generated according to method a) (FIG. 4A).

Microcapsules-based SAG assemblies generated according to methods c) and d) were significantly smaller than assemblies for FACS-based prokaryote SAGs (FIG. 4B). This provided further evidence that most DNA-containing microcapsules did not contain genomes of Prokarya.

Microcapsules-based SAG libraries generated according to methods c)-d) contained a larger fraction of viral genome assemblies deemed as “complete” or “high quality”, relative to FACS-based viral SAGs generated according to the method b (FIG. 4C).

Microcapsules-based SAG libraries generated according to methods c)-d) recruited a larger number of viral metagenome reads relative to FACS-based viral SAGs generated according to the method b (FIG. 4D).

Finally, many viral genome assemblies generated according to methods c)-d) had no similarity to assemblies obtained from the viral metagenome (FIG. 7). These results demonstrate the recovery of viral genomes that could not be recovered by either metagenome assembly or FACS-based genomics of individual viral particles.

The larger assemblies (FIG. 4B), higher fraction of complete and high-quality assemblies (FIG. 4C), higher recruitment of metagenome reads (FIG. 4D) and higher fraction of microcapsules containing amplifiable DNA (results not shown) in the cryopreserved sample as compared to fresh sample means that cryopreservation increased the number of viruses from which DNA could be amplified and improved the quality of the obtained viral genome assemblies. Potential causes of this unexpected effect of cryopreservation include but are not limited to: i) viral capsid damage by sample freezing and thawing, leading to the exposure of additional viral DNA to amplification reagents; and ii) inhibition of DNA degradation by environmental nucleases by the presence of EDTA in the cryopreservation buffer.

To create the viral metagenome from the Gulf of Maine for the results shown in FIG. 4D, a 40 L sample of seawater was collected with a Niskin bottle from 1 meter depth in Boothbay (43.84° N, 69.64° W) on Monday Apr. 18th, 2022. Seawater was filtered through a 0.2 μm mesh size filter and concentrated to 250 ml using a REXEED 30 kMWCO tangential flow filter system (Asahi Kasei Medical) (18). Concentrated viral samples were then stored at 4° C. overnight. The following morning, the concentrated sample was further concentrated to ˜1.5 ml final volume (˜30,000×concentration) using Amicon 100 kMWCO ultracentrifugation filters (Millipore Sigma) in a refrigerated centrifuge. The final sample was resuspended off the filter in deionized water and stored in a sterile, 1.5 ml Eppendorf tube. The DNA was extracted from a 200 uL aliquot of the final concentrated sample (equivalent to ˜7 L of the original sample) using previously described protocols (21). Libraries for DNA sequencing were created with Nextera XT (Illumina) reagents following manufacturer's instructions except for purification steps, which were done with column cleanup kits (QIAGEN) and library size selection, which was done with BluePippin (Sage Science, Beverly, MA) with a target size of 500±50 bp. DNA concentration measurements were performed with Quant-iT™ dsDNA Assay Kits (Thermo Fisher Scientific) following manufacturer's instructions. Libraries were sequenced with NextSeq 2000 (Illumina) in 2×150 bp mode. The obtained sequence reads were quality-trimmed with Trimmomatic v0.3260 using the following settings: -phred33 LEADING:0 TRAILING:5 SLIDINGWINDOW:4:15 MINLEN:36. Reads matching the H. sapiens reference assembly GRCh38 and a local database of reagent contaminants16 (≥95% identity of ≥100 bp alignments) as well as low complexity reads (containing <5% of any nucleotide) were removed. Reads were assembled into contigs using metaSPades (19) using default parameters. Metagenomic fragment recruitment of BBH viral metagenomic reads to SAG, microcapsule and metagenomic assemblies was conducted using CoverM (-min-read-percent-identity 95-min-read-aligned-percent 50).

### Example 3: Recovery of Viral DNA Sequences from Sediment Sample

Gulf of Maine coastal sediment sample was collected from 9-10 cm depth on Mar. 24, 2022, in Boothbay Harbor (43.99° N 69.65° W), mixed 1:100 with autoclaved seawater, amended with 5% glycerol and 1×pH 8 Tris-EDTA buffer (final concentrations) and stored at −80° C. until further processing. Microcapsule data were generated by: (i) cryopreservation of the sample; (ii) generation of semi-permeable hydrogel microcapsules filled with random micro-aliquots of the sample (after thawing the sample); (iii) viral particle lysis and genomic DNA amplification inside the microcapsules; (iv) separation of individual microcapsules that contain amplified DNA into microplate wells; (v) lysis of microcapsules and a second round of DNA amplification in microplate wells; and (vi) production of uniquely barcoded sequencing libraries inside microplate wells. The methods did not include steps of removal of cellular material prior to encapsulation and digestion of naked DNA with a DNAse treatment inside microcapsules.

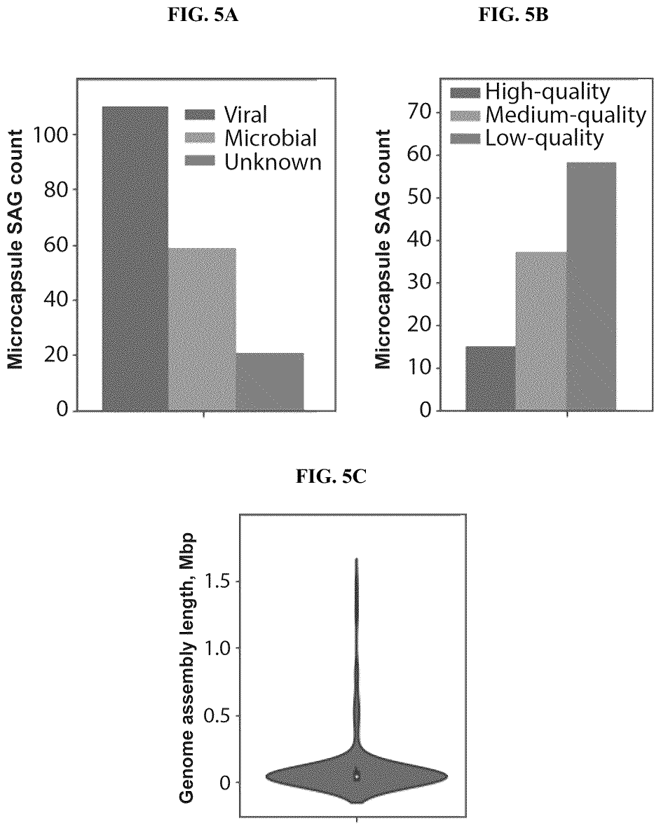

The encapsulation of samples, particle and microcapsule lysis, DNA amplification, and the production of sequencing libraries were performed as described in Example 2. Results are shown in FIGS. 5A-5C. A large fraction of microcapsule-based SAGs was of viral origin (FIG. 5A). High quality viral genome sequences were obtained from the marine sediment samples (FIG. 5B). Most microcapsule-based SAG assemblies were smaller than 200 kilobases, which is expected from free viral particles and fragments of extracellular DNA, while larger assemblies are expected from intact cells (FIG. 5C). These results demonstrate the genomic sequencing of individual viral particles and extracellular, non-viral DNA molecules from a from a complex, sediment sample.

### Example 4: Recovery of Viral DNA Sequences from Deep Ocean Seawater

Two samples of deep ocean seawater were collected: 1) polar, from 300 m depth at 88.71° N 46.09° E on Sep. 8, 2018; and 2) tropical, from 3,000 m depth at 0° N 180° E on May 12, 2016. Field samples were amended with 5% glycerol and 1×pH 8 Tris-EDTA buffer (final concentrations) and stored at −80° C. until further processing. Microcapsules were generated by: (i) cryopreservation of the sample; (ii) generation of semi-permeable hydrogel microcapsules filled with random micro-aliquots of the sample (after thawing the sample); (iii) viral particle lysis and genomic DNA amplification inside the microcapsules; (iv) separation of individual microcapsules that contain amplified DNA into microplate wells; (v) lysis of microcapsules and a second round of DNA amplification in microplate wells; and (vi) production of uniquely barcoded sequencing libraries inside microplate wells. Samples were not treated to remove cellular material prior to encapsulation or to digest naked DNA with a DNAse treatment inside capsules.

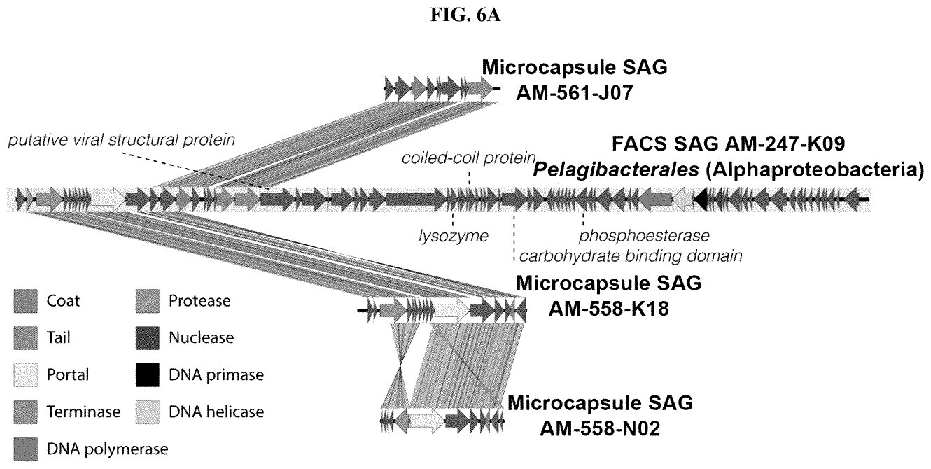

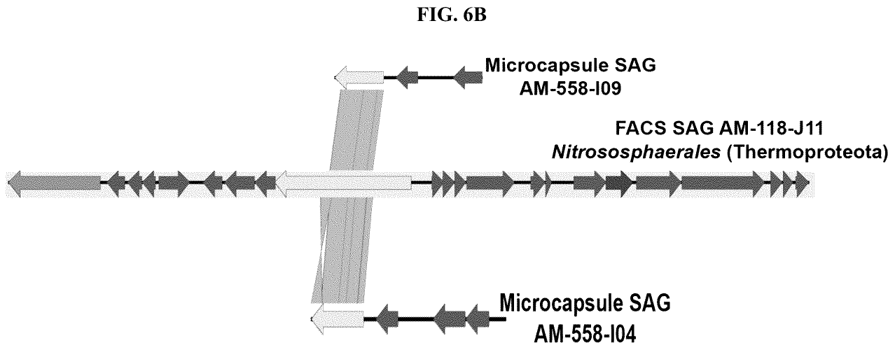

The encapsulation of samples, particle lysis, DNA amplification, and the production of sequencing libraries were performed as described in Example 2. Results are shown in FIGS. 6A-6B. These results demonstrate the capacity of the microcapsule-based genomics to recover genomic sequences of individual, free particles of viruses that are capable of infecting microbial cells in the same environment.

### Example 5: Quantification of Viral Particles in Samples

Viral particles were quantified in the samples generated according to: methods c) and d) of Example 2; the method of Example 3; and the method of Example 4.

The quantification of specific viral particles was performed as follows. The average volume of newly formed capsules, V_{c}, was determined using the built-in image analysis system of the ONYX (Droplet Genomics) instrument. V_{c} was maintained between 3.5 and 8.0 pL. Average volume of the analyzed sample in a single microcapsule, V_{cs} was estimated as follows: V_{cs=}V_{c}×Rₛ/(Rₛ+R_{PEGDA}+R_{D}), where Rₛ, R_{PEGDA} and R_{D} are on-chip flow rates of the sample, PEGDA and dextran solutions during microcapsule generation. The fraction of microcapsules containing DNA, F_{DNA}, was determined flow-cytometrically after an in-capsule DNA amplification by WGA-X and staining with a nucleic acid stain SYTO-9 (ThermoFisher Scientific). The fraction of DNA-containing microcapsules containing viral particles, Fᵥᵢᵣₐₗ, was determined using genomic sequencing and bioinformatics methods described above. Subsequently, the abundance of virus-like particles of a particular type, Aᵥᵢᵣₐₗ, was estimated as follows: Aᵥᵢᵣₐₗ=(Fᵥᵢᵣₐₗ×F_{DNA}×Dₛ)/V_{c}, where Ds is sample dilution prior to microcapsule generation. For this method to produce reasonably accurate estimates, F_{DNA} must be maintained below 10%.

Using this approach, we obtained the following abundance estimates of virus-like particles with <50% genes found to be host-associated by CheckV (17):
Gulf of Maine seawater:
   Method c) of Example 2 (a freshly prepared sample): 1.6×10⁶ mL⁻¹
   Method d) of Example 2 (a cryopreserved sample): 8.6×10⁶ mL⁻¹
Polar deep ocean seawater—method of Example 4 (a cryopreserved sample): 2.9×10⁶ mL⁻¹
Tropical deep ocean seawater—method of Example 4 (a cryopreserved sample): 2.3×10⁶ mL⁻¹

The current knowledge of viral abundance in surface ocean suggests typical counts of between 10⁶ and 10⁸ viral particles per mL seawater (20), which is in good agreement with the viral abundance estimate using the microencapsulation approach on the cryopreserved Gulf of Maine sample. For comparison, the count of virus-like particles with <50% genes found to be host-associated by CheckV (17) obtained using the FACS-based approach in the cryopreserved Gulf of Maine Seawater sample was 1.2×10⁶ mL⁻¹.

The lower estimate of viruses in the fresh Gulf of Maine seawater sample suggests that cryopreservation enabled DNA sequence recovery from a larger fraction of marine viruses. Due to the potential inefficiencies in viral particle lysis, nucleic acid amplification and other steps in the workflow, these estimates should be considered conservative and may be further improved by spiking the analyzed samples with relevant internal standards.

### REFERENCES

1. B. L. Hurwitz, A. Ponsero, J. Thornton, J. M. U'Ren, Phage hunters: Computational strategies for finding phages in large-scale ‘omics datasets. Virus Research 244, 110-115 (2018).
2. S. Roux, J. B. Emerson, E. A. Eloe-Fadrosh, M. B. Sullivan, Benchmarking viromics: An in silico evaluation of metagenome-enabled estimates of viral community composition and diversity. PeerJ 2017, (2017).
3. J. Warwick-Dugdale et al., Long-read viral metagenomics captures abundant and microdiverse viral populations and their niche-defining genomic islands. PeerJ 2019, (2019).
4. Y. Benjamini, T. P. Speed, Summarizing and correcting the GC content bias in high-throughput sequencing. Nucleic Acids Research 40, (2012).
5. J. Beaulaurier et al., Assembly-free single-molecule sequencing recovers complete virus genomes from natural microbial communities. Genome Res 30, 437-446 (2020).
6. W. H. Wilson et al., Genomic exploration of individual giant ocean viruses. ISME Journal 11, 1736-1745 (2017).
7. R. Stepanauskas et al., Improved genome recovery and integrated cell-size analyses of individual uncultured microbial cells and viral particles. Nat Commun 8, 84 (2017).
8. F. Martinez-Hernandez et al., Single-virus genomics reveals hidden cosmopolitan and abundant viruses. Nat Commun 8, 15892 (2017).
9. F. Azam, OCEANOGRAPHY: Microbial Control of Oceanic Carbon Flux: The Plot Thickens. Science 280, 694-696 (1998).
10. A. M. Klein et al., Droplet barcoding for single-cell transcriptomics applied to embryonic stem cells. Cell 161, 1187-1201 (2015).
11. E. Z. Macosko et al., Highly parallel genome-wide expression profiling of individual cells using nanoliter droplets. Cell 161, 1202-1214 (2015).
12. F. Lan, B. Demaree, N. Ahmed, A. R. Abate, Single-cell genome sequencing at ultra-high-throughput with microfluidic droplet barcoding. Nature Biotechnology 35, 640-646 (2017).
13. G. Leonaviciene, K. Leonavicius, R. Meskys, L. Mazutis, Multi-step processing of single cells using semi-permeable capsules. Lab Chip 20, 4052-4062 (2020).
14. A. Bankevich, S. Nurk, D. Antipov, A. A. Gurevich, M. Dvorkin, A. S. Kulikov, V. M. Lesin, S. I. Nikolenko, S. Pham, A. D. Prjibelski, A. V. Pyshkin, A. V. Sirotkin, N. Vyahhi, G. Tesler, M. A. Alekseyev, P. A. Pevzner, SPAdes: A new genome assembly algorithm and its applications to single-cell sequencing. Journal of Computational Biology 19:455-477 (2012).
15. T. Seemann, Prokka: rapid prokaryotic genome annotation. Bioinformatics 30:2068-2069 (2014).
16. Guo J, Bolduc B, Zayed A A, Varsani A, Dominguez-Huerta G, Delmont T O, Pratama A A, Gazitua M C, Vik D, Sullivan M B, Roux S. 2021. VirSorter2: a multi-classifier, expert-guided approach to detect diverse DNA and RNA viruses. Microbiome 9:37.
17. Nayfach S, Camargo A P, Schulz F, Eloe-Fadrosh E, Roux S, Kyrpides N C. 2021. CheckV assesses the quality and completeness of metagenome-assembled viral genomes. Nat Biotechnol 39:578-585.
18. Hill V R, Polaczyk A L, Hahn D, Narayanan J, Cromeans T L, Roberts J M, Amburgey J E. 2005. Development of a rapid method for simultaneous recovery of diverse microbes in drinking water by ultrafiltration with sodium polyphosphate and surfactants. Appl Environ Microbiol 71:6878-6884.
19. Nurk S, Meleshko D, Korobeynikov A, Pevzner P A. 2017. metaSPAdes: a new versatile metagenomic assembler. Genome Res 27:824-834.
20. Wigington C H, Sonderegger D, Brussaard C P, Buchan A, Finke J F, Fuhrman J A, Lennon J T, Middelboe M, Suttle C A, Stock C, Wilson W H, Wommack K E, Wilhelm S W, Weitz J S (2016) Re-examination of the relationship between marine virus and microbial cell abundances. Nat Microbiol 1:15024
21. Thurber R V, Haynes M, Breitbart M, Wegley L, Rohwer F (2009) Laboratory procedures to generate viral metagenomes. Nat Protoc 4:470-483

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A method of sequencing individual viral genomes comprising:
(i) encapsulating aliquots of a liquid sample in semi-permeable microcapsules to generate a plurality of encapsulated aliquots collectively comprising a plurality of viral genomes;
(ii) exposing the plurality of encapsulated aliquots to amplification conditions to amplify genomic material within the encapsulated aliquots; and
(iii) sequencing the amplified genomic material of the encapsulated aliquots.

2. The method of claim 1, wherein the semi-permeable microcapsules are semi-permeable hydrogel microcapsules.

3. The method of claim 1, further comprising separating encapsulated aliquots containing amplified genomic material from encapsulated aliquots lacking amplified genomic material between steps (ii) and (iii).

4. The method of claim 3, wherein the aliquots containing amplified genomic material are separated into individual microwells.

5. The method of claim 1, wherein the volume of the aliquots encapsulated by the individual semi-permeable microcapsules is between 1-100 pL.

6. The method of claim 1, wherein the liquid sample is a liquid biological sample, optionally wherein the liquid biological sample is isolated from a multicellular organism, wherein the multicellular organism is an animal, a fungus, or a plant or wherein the liquid biological sample comprises blood, saliva, or mucous.

7.-8. (canceled)

9. The method of claim 1, wherein the liquid sample is a liquid environmental sample, optionally wherein the liquid environmental sample is a seawater sample, a lake water sample, a river sample, or a wastewater sample.

10. (canceled)

11. The method of claim 1, further comprising:
enriching for extracellular genetic elements in the liquid sample prior to encapsulating in step (i);
diluting the liquid sample to increase the probability that no more than one genomic molecule is encapsulated in each semi-permeable microcapsule;
or a combination thereof.

12. (canceled)

13. The method of claim 1, wherein the liquid sample is cryopreserved and thawed prior to step (i).

14. The method of claim 1, wherein the liquid sample subject to encapsulation in step (i) is a raw liquid sample.

15. The method of claim 1, wherein the liquid sample is generated by dispersing a solid sample into a liquid, optionally wherein the sample is cryopreserved after dispersal and thawed prior to step (i).

16. The method of claim 15, wherein the solid sample is a solid biological sample, optionally wherein the solid biological sample is isolated from a multicellular organism, wherein the multicellular organism is an animal, a fungus, or a plant, or wherein the solid biological sample is fecal material or skin.

17.-18. (canceled)

19. The method of claim 15, wherein the solid sample is a solid environmental sample, optionally wherein the solid environmental sample is a surface swab, a soil sample, a rock sample, or a marine sediment sample.

20. (canceled)

21. The method of claim 1, further comprising enriching for extracellular genetic elements in the sample prior to encapsulating in step (i), optionally wherein the enriching comprises separating cellular material from extracellular genetic elements.

22. (canceled)

23. The method of claim 1, further comprising:
flocculation, filtration, flow cytometry, or a combination thereof;
exposing the plurality of encapsulated aliquots to a DNase prior to exposure to amplification conditions in step (ii);
enriching for semi-permeable microcapsules containing amplified genetic elements prior to sequencing in step (iii); or
any combination thereof.

24.-26. (canceled)

27. The method of claim 1, further comprising positioning microcapsules containing amplified genetic elements in individual wells of a microplate prior to sequencing in step (iii), optionally wherein the method further comprises lysing the microcapsules in the individual wells of the microplate and/or re-amplifying the amplified genetic elements in the individual wells of the microplate prior to step (iii).

28.-29. (canceled)

30. The method of claim 1, further comprising barcoding amplified genetic elements prior to sequencing in step (iii), optionally wherein the amplified genetic elements are barcoded in individual wells of a microplate.

31. (canceled)

32. The method of claim 1, wherein the plurality of viral genomes of (i) comprises DNA viral genomes, optionally wherein the DNA viral genomes comprise single-stranded DNA viral genomes and/or wherein the DNA viral genomes comprise genomes that are smaller than 30 kbp.

33.-34. (canceled)

35. A method of determining the viral load of a liquid sample comprising:
(i) encapsulating aliquots of the liquid sample in individual semi-permeable microcapsules to generate a plurality of encapsulated aliquots, wherein the total volume of liquid sample encapsulated within the random aliquots is known;
(ii) exposing the plurality of encapsulated aliquots to amplification conditions to amplify genomic material within the encapsulated aliquots;
(iii) sequencing the amplified genomic material of the encapsulated aliquots to identify and quantify individual viral genomes in the plurality of encapsulated aliquots; and
(iv) calculating viral load in the sample using the total volume of sample encapsulated within the random aliquots in (i) and the quantity of individual viral genomes of (iii).

36.-68. (canceled)

69. A method of monitoring the evolution of a viral genome comprising:
(i) encapsulating, at a first time point, random aliquots of a first liquid sample in semi-permeable microcapsules to generate a plurality of encapsulated aliquots collectively comprising a plurality of viral genomes;
(ii) exposing the plurality of encapsulated aliquots to amplification conditions to amplify genomic material within the encapsulated aliquots;
(iii) sequencing the amplified genomic material of the encapsulated aliquots, thereby determining the sequences of the viral genomes in the plurality of viral genomes;
(iv) identifying mutations in the viral genomes determined in (iii) by comparing their sequences with those of previously identified viral genomes; and
(v) iteratively repeating steps (i)-(iv) at later time points and with additional liquid samples.

70.-99. (canceled)
